(19) United States
(12) Patent Application Publication
(10) Pub. No.: US 2021/0045716 A1
(43) Pub. Date: Feb. 18, 2021

(54) METHOD AND SYSTEM FOR PROVIDING INTERACTION WITH A VISUAL ARTIFICIAL INTELLIGENCE ULTRASOUND IMAGE SEGMENTATION MODULE

(71) Applicant: GE Precision Healthcare LLC, Wauwatosa, WI (US)

(72) Inventors: Carmit Shiran, Zichron Yakkov (IL); Dani Pinkovich, Haifa (IL); Sagi Schein, Haifa (IL); Doron Shaked, Haifa (IL); Antonio Fabian Fermoso, Madrid (ES); Naomi Sato, Tokyo (JP)

(21) Appl. No.: 16/539,339

(22) Filed: Aug. 13, 2019

(51) Int. Cl. A61B 8/08; G06T 7/11; G06N 3/02; G06N 20/00

(52) U.S. Cl. CPC A61B 8/5223; A61B 8/483; A61B 8/5207; G06T 7/0012; G06N 3/02; G06N 20/00; G06T 7/11

(57) ABSTRACT

A system and method for facilitating interaction by an ultrasound operator with an artificial intelligence segmentation module configured to identify and track biological and/or artificial structures in ultrasound images is provided. The method includes acquiring an ultrasound image and segmenting the ultrasound image with artificial intelligence to identify structure(s) in the ultrasound image. The method includes labeling the structure(s) in the ultrasound image to create a labeled ultrasound image. The method includes presenting the labeled ultrasound image at a display system. The method includes receiving a user selection of at least one target corresponding with at least one labeled structure. The method includes tracking the selected target(s) by identifying the selected target(s) in subsequently acquired ultrasound images.

### FIELD

Certain embodiments relate to ultrasound imaging. More specifically, certain embodiments relate to a method and system providing an interface for an ultrasound operator to interact with an artificial intelligence segmentation module configured to identify and track biological and/or artificial structures in ultrasound images.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

During an ultrasound-based regional anesthesia procedure, an anesthesiologist may operate both an ultrasound system and the insertion and navigation of a needle to its destination such that an appropriate amount of anesthetic medium may be administered to the destination (e.g., a designated nerve). Accordingly, the anesthesiologist may provide simultaneous visual attention to the ultrasound system display and the patient such that the anesthesiologist may track targets (e.g., the needle, the designated nerve, etc.) while navigating the needle around critical organs (e.g., vessels) to the destination. In order to provide such simultaneous visual attention, anesthesiologists often position the ultrasound system on an opposite side of the patient such that both the ultrasound system display and the patient are kept in a same field of view. Since both hands of the anesthesiologist are typically occupied and the display may be out of reach, it can be difficult for an anesthesiologist to specify actions, select objects, and/or select locations on an ultrasound system display during the procedure.

Artificial intelligence processing of ultrasound images and/or video is often applied to process the images and/or video to assist an ultrasound operator or other medical personnel viewing the processed image data with providing a diagnosis. However, artificial intelligence processing is typically static in nature. Specifically, a computer may receive an image and/or video, process the image and/or video in a pre-defined manner using the artificial intelligence, and output a result (e.g., a processed image or video that may be manipulated by a user). The static nature of the artificial intelligence processing provides a lack of dynamic adaptability of the processing for different functionality as desired by a user, thereby limiting the use of the artificial intelligence processing to a particular application.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for facilitating interaction by an ultrasound operator with an artificial intelligence segmentation module configured to identify and track biological and/or artificial structures in ultrasound images, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to facilitate interaction by an ultrasound operator with an artificial intelligence segmentation module configured to identify and track biological and/or artificial structures in ultrasound images, in accordance with various embodiments.

FIG. 2 is a display of an exemplary ultrasound image provided by an artificial intelligence segmentation module configured to identify and track biological and/or artificial structures based on ultrasound operator interaction, in accordance with various embodiments.

FIG. 3 illustrates screenshots of a series of displays over time of exemplary ultrasound images provided by an artificial intelligence segmentation module configured to identify and track biological and/or artificial structures based on ultrasound operator interaction, in accordance with various embodiments.

FIG. 4 is a flow chart illustrating exemplary steps that may be utilized for identifying and tracking biological and/or artificial structures by an artificial intelligence segmentation module based on ultrasound operator interaction, in accordance with various embodiments.

### DETAILED DESCRIPTION

Certain embodiments may be found in a method and system for facilitating interaction by an ultrasound operator with an artificial intelligence segmentation module configured to identify and track biological and/or artificial structures in ultrasound images. Various embodiments have the technical effect of dynamically identifying one or more biological and/or artificial structures as targets to track via an artificial intelligence segmentation module by allowing an ultrasound operator to interact with the artificial intelligence segmentation module to provide identification and/or tracking instructions. Aspects of the present disclosure have the technical effect of facilitating ultrasound operator interaction with an artificial intelligence segmentation module without having to touch a control panel or touchscreen display of an ultrasound system (e.g., voice and/or probe controls).

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

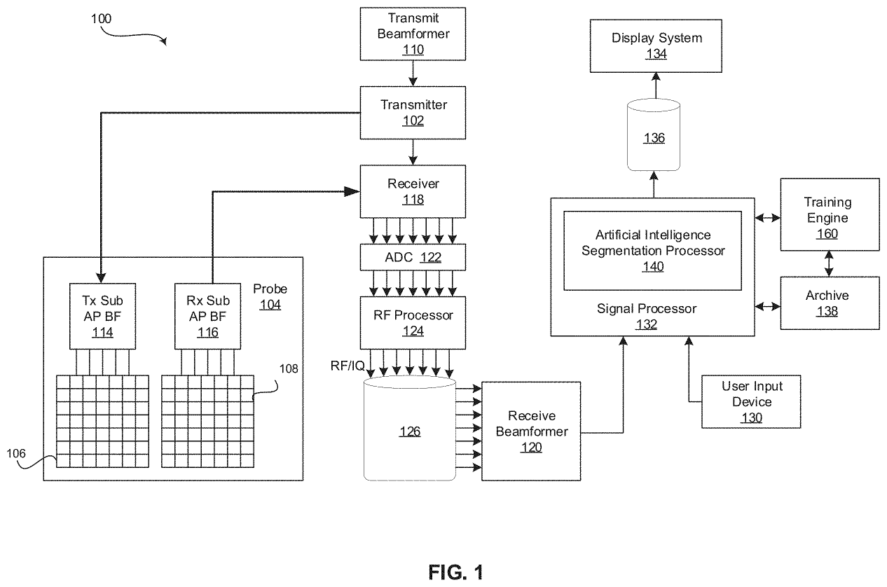

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to facilitate interaction by an ultrasound operator with an artificial intelligence segmentation module 140 configured to identify and track biological and/or artificial structures in ultrasound images 200, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, an archive 138, and a training engine 160.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, interact with an artificial intelligence segmentation processor to select tracking targets, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display. As another example, user input device 130 may include an accelerometer, gyroscope, and/or magnetometer attached to and/or integrated with the probe 104 to provide gesture motion recognition of the probe 104, such as to identify one or more probe compressions against a patient body, a pre-defined probe movement or tilt operation, or the like. Additionally and/or alternatively, the user input device 130 may include image analysis processing to identify probe gestures by analyzing acquired image data.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an artificial intelligence segmentation processor 140 and may be capable of receiving input information from a user input device 130 and/or archive 138, generating an output displayable by a display system 134, and manipulating the output in response to input information from a user input device 130, among other things. The signal processor 132 and artificial intelligence segmentation processor 140 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an artificial intelligence segmentation processor 140 that comprises suitable logic, circuitry, interfaces and/or code that may be operable to analyze acquired ultrasound images to identify, segment, label, and track biological and/or artificial structures. The biological structures may include, for example, nerves, vessels, organ, tissue, or any suitable biological structures. The artificial structures may include, for example, a needle, an implantable device, or any suitable artificial structures. The artificial intelligence segmentation processor 140 may include artificial intelligence image analysis algorithms, one or more deep neural networks (e.g., a convolutional neural network) and/or may utilize any suitable form of artificial intelligence image analysis techniques or machine learning processing functionality configured to analyze acquired ultrasound images to identify, segment, label, and track biological and/or artificial structures.

The artificial intelligence segmentation processor 140 may comprise suitable logic, circuitry, interfaces and/or code that may be operable to analyze acquired ultrasound images to identify and segment biological and/or artificial structures. In various embodiments, the artificial intelligence segmentation processor 140 may be provided as a deep neural network that may be made up of, for example, an input layer, an output layer, and one or more hidden layers in between the input and output layers. Each of the layers may be made up of a plurality of processing nodes that may be referred to as neurons. For example, the artificial intelligence segmentation processor 140 may include an input layer having a neuron for each pixel or a group of pixels from a scan plane of an anatomical structure. The output layer may have a neuron corresponding to a plurality of pre-defined biological and/or artificial structures. As an example, if performing an ultrasound-based regional anesthesia procedure, the output layer may include neurons for a brachial plexus nerve bundle, the axillary artery, beveled regions on anesthetic needles, and the like. Other ultrasound procedures may utilize output layers that include neurons for nerves, vessels, bones, organs, needles, implantable devices, or any suitable biological and/or artificial structure. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the artificial intelligence segmentation processor 140 deep neural network (e.g., convolutional neural network) may identify biological and/or artificial structures in ultrasound image data with a high degree of probability.

In certain embodiments, the artificial intelligence segmentation processor 140 may be configured to identify and segment biological and/or artificial structures based on a user instruction via the user input device 130. For example, the artificial intelligence segmentation processor 140 may be configured to interact with a user via the user input device 130 to receive instructions for searching the ultrasound image. As an example, a user may provide a voice command, probe gesture, button depression, or the like that instructs the artificial intelligence segmentation processor 140 to search for a particular structure and/or to search a particular region of the ultrasound image.

The artificial intelligence segmentation processor 140 may comprise suitable logic, circuitry, interfaces and/or code that may be operable to label the identified and segmented biological and/or artificial structures. For example, the artificial intelligence segmentation processor 140 may label the identified and segmented structures identified by the output layer of the deep neural network. The labels may include colorizing the pixels of the segmented structure, outlining the edges of the segmented structure, identifying the segmented structure by a number or letter, or any suitable label for drawing attention to one or more structures identified and segmented by the artificial intelligence segmentation processor 140. In various embodiments, the label type provided by the artificial intelligence segmentation processor 140 may correspond with a confidence level of the identified structure. For example, a colorized structure may correspond with a highest level of confidence, a structure outlined with solid lines may correspond with a middle level of confidence, and a structure outlined with dashed lines may correspond with a low level of confidence. The labels may be overlaid on the ultrasound image and presented at the display system 134.

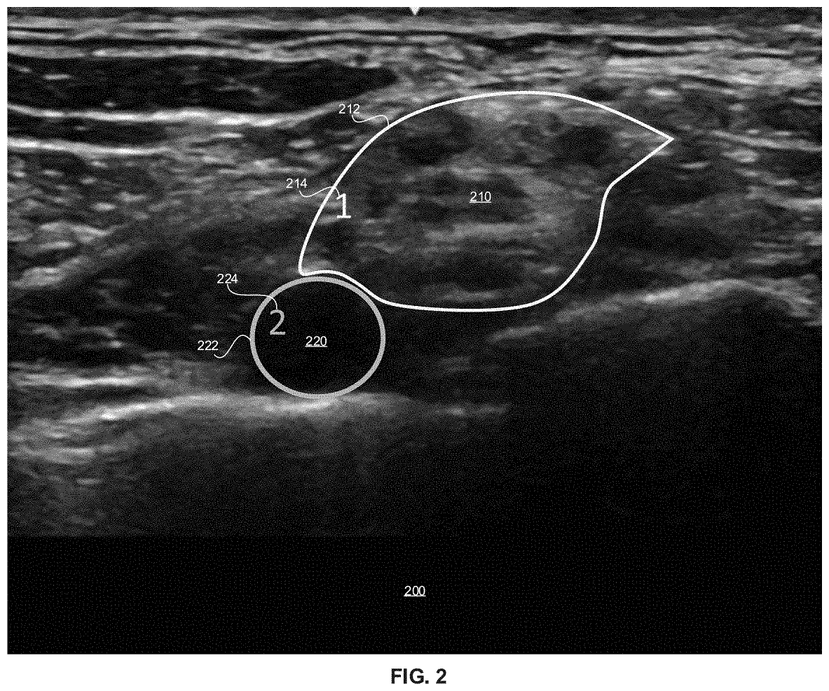

FIG. 2 is a display of an exemplary ultrasound image 200 provided by an artificial intelligence segmentation module 140 configured to identify and track biological and/or artificial structures 210, 220 based on ultrasound operator interaction, in accordance with various embodiments. Referring to FIG. 2, the ultrasound image 200 may comprise labels 212, 214, 222, 224 identifying structures 210, 220 identified and segmented by the artificial intelligence segmentation module 140. For example, the labels may include a solid line 212, 222 outlining the outer edges of the identified and segmented structures 210, 220. As another example, the labels may include numbers 214, 224, letters, text, or the like corresponding with the identified and segmented structures 210, 220. The labels 212, 214, 222, 224 may be colored, such as to further distinguish multiple identified and segmented structures 210, 220 in the ultrasound image 200. Other labels not shown in FIG. 2 may include colorization of the pixels of the structures 210, 220, dashed lines outlining the identified and segmented structures 210, 220, or the like. In various embodiments, different label types may correspond to different confidence levels associated with the identified and segmented structures 210, 220.

In an exemplary embodiment, an ultrasound operator may interact with the artificial intelligence segmentation processor 140 via the user input device 130 based on the presented labeled ultrasound image. For example, FIG. 2 illustrates a brachial plexus nerve 210 outlined 212 in a first color and having an associated number label of “1”. FIG. 2 further illustrates a vessel 220 outlined 222 in a second color and having an associated number label of “2”. The ultrasound operator may provide a voice command to select the brachial plexus nerve 210 to be tracked by stating: “select nerve,” “select brachial plexus,” “select organ 1,” “select yellow segment,” or any suitable voice command. The ultrasound operator may provide a voice command to deselect the vessel 220 by stating: “deselect vessel,” “deselect organ 2,” “deselect red segment,” or any suitable voice command. For example, an ultrasound operator may observe a problem with an identified structure, such as a needle, in the ultrasound image 200 and instruct the artificial intelligence segmentation module 140 to forget the incorrect needle and look for the needle in the location indicated by the user. In response, the artificial intelligence segmentation module 140 may modify the image acquisition parameters and/or the image recognition algorithm to improve the identification and segmentation result. The artificial intelligence segmentation module 140 may, for example, place a “no needle” in the region where it thought the needle was but the ultrasound operator indicated was incorrect.

In an exemplary embodiment, the ultrasound operator may provide a voice command to search for additional structures by stating: “search to the left of organ 2 for needle” or any suitable voice command. For example, the ultrasound operator may indicate a specific region of interest in the image and the artificial intelligence segmentation module 140 can then classify that object (e.g., kidney in an abdominal image). As another example, the ultrasound operator may state: “find me the aorta in the image” and the artificial intelligence segmentation module 140 may find all the arteries in the image, separate the arteries from veins and other anatomies, and highlight the arteries or highlight the aorta if the artificial intelligence segmentation module 140 can differentiate the aorta from smaller arteries.

In various embodiments, the ultrasound operator may provide a voice command to track multiple targets merged together by stating: “track union of organ 1 and organ 2” or any suitable voice command. As another example, the ultrasound operator may operate controls on the probe 104 or a control panel to toggle to between and select a structure 210, 220 to track. In certain embodiments, the ultrasound operator may operate the probe 104 as a user input device 130 by gesture recognition, such as tilting the probe 104, providing a double compression movement against a patient, or any suitable pre-defined movement, position, and/or orientation associated with an action to toggle between and select a structure 210, 220 to track. In various embodiments, the artificial intelligence segmentation module 140 may alternately highlight the various structures 210, 220 (referred to as a rolling highlight) and the ultrasound operator may provide an input via the user input module 130 to select a currently highlighted structure 210, 220.

Referring again to FIG. 1, the artificial intelligence segmentation processor 140 may comprise suitable logic, circuitry, interfaces and/or code that may be operable to track selected biological and/or artificial structures 210, 220. For example, the artificial intelligence segmentation processor 140 may be configured to interact with a user via the user input device 130 to receive instructions for selecting or disregarding labeled structures 210, 220 to be tracked in subsequently acquired ultrasound images. The selection of a labeled structure 210, 220 identifies a target to track in subsequent ultrasound images. As an example, a user may provide a voice command, probe gesture, button depression, or the like that instructs the artificial intelligence segmentation processor 140 to select labeled structures to track and/or deselect labeled structures 210, 220 from being identified in subsequent ultrasound images as described above with reference to FIG. 2. The selection may include selecting multiple targets to be tracked and/or instructing the artificial intelligence segmentation processor 140 to merge the targets to be tracked in subsequent ultrasound images. The artificial intelligence segmentation processor 140 may modify the image identification, segmentation, labeling, and/or tracking parameters dynamically in response to the user instructions received via the user input device 130.

In various embodiments, the artificial intelligence segmentation processor 140 may be configured to provide user feedback based on the location of the tracked target. For example, the artificial intelligence segmentation processor 140 may provide audible, visual, and/or physical feedback if a tracked target is approaching an image boundary. The audible feedback may be an alarm, warning message, or any suitable audible feedback. The visual feedback may include a visual message, flashing label, or any suitable visual feedback. The physical feedback may include causing the probe to vibrate, or any suitable physical feedback.

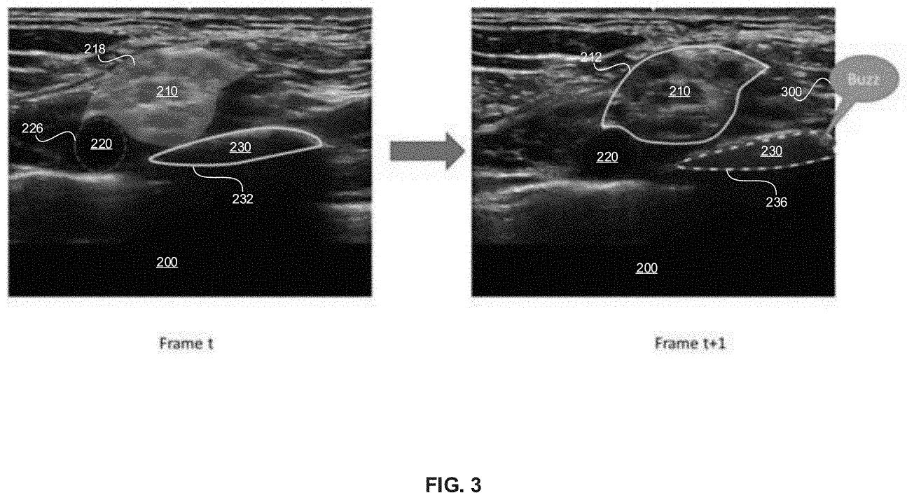

FIG. 3 illustrates screenshots of a series of displays over time of exemplary ultrasound images 200 provided by an artificial intelligence segmentation module 140 configured to identify and track biological and/or artificial structures 210, 200, 230 based on ultrasound operator interaction, in accordance with various embodiments. Referring to FIG. 3, a first ultrasound image 200 at a first time (t) and a second ultrasound image 200 at a second time (t+1) may comprise labels 212, 218, 226, 232, 236 identifying structures 210, 220, 230 identified and segmented by the artificial intelligence segmentation module 140. For example, the labels may include a solid line 212, 232 outlining the outer edges of the identified and segmented structures 210, 230. As another example, the labels 212, 218, 226, 232, 236 may be colored, such as to further distinguish multiple identified and segmented structures 210, 220, 230 in the ultrasound image 200. The labels may include colorization of the pixels 218 of the structure 210, dashed lines 226, 236 outlining the identified and segmented structures 220, 230, or the like. In various embodiments, different label types may correspond to different confidence levels associated with the identified and segmented structures 210, 220, 230. In certain embodiments, the artificial intelligence segmentation processor 140 may provide feedback 300 if a tracked target 230 is approaching an image boundary. For example, the feedback 300 may be audible, visual, physical, and/or any suitable feedback to alert a user of a pre-defined condition present in the ultrasound image 200.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present ultrasound images and/or any suitable information. For example, the ultrasound images presented at the display system 134 may include labels, tracking identifiers, and or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound image data, labeled ultrasound images, identification instructions, segmentation instructions, labeling instructions, and tracking instructions, for example.

Still referring to FIG. 1, the training engine 160 may comprise suitable logic, circuitry, interfaces and/or code that may be operable to train the neurons of the deep neural network(s) of the artificial intelligence segmentation module 140. For example, the artificial intelligence segmentation module 140 may be trained to automatically identify and segment biological and/or artificial structures provided in an ultrasound scan plane. For example, the training engine 160 may train the deep neural networks of the artificial intelligence segmentation module 140 using databases(s) of classified ultrasound images of various structures. As an example, the artificial intelligence segmentation module 140 may be trained by the training engine 160 with ultrasound images of particular biological and/or artificial structures to train the artificial intelligence segmentation module 140 with respect to the characteristics of the particular structure, such as the appearance of structure edges, the appearance of structure shapes based on the edges, the positions of the shapes relative to landmarks in the ultrasound image data, and the like. In an exemplary embodiment, the structures may include a brachial plexus nerve bundle, the axillary artery, beveled regions on anesthetic needles, and/or any suitable organ, nerve, vessel, tissue, needle, implantable device, or the like. The structural information may include information regarding the edges, shapes, and positions of organs, nerves, vessels, tissue, needles, implantable devices, and/or the like. In various embodiments, the databases of training images may be stored in the archive 138 or any suitable data storage medium. In certain embodiments, the training engine 160 and/or training image databases may be external system(s) communicatively coupled via a wired or wireless connection to the ultrasound system 100.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

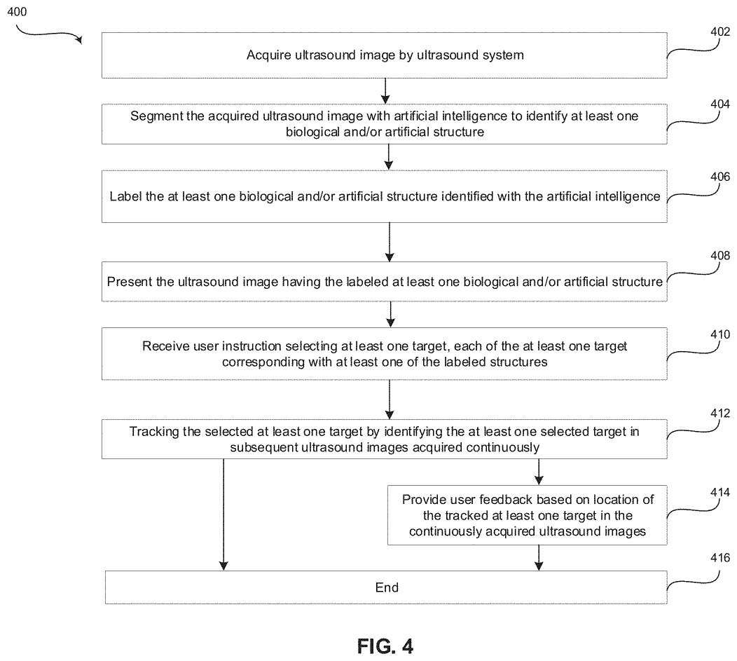

FIG. 4 is a flow chart 400 illustrating exemplary steps 402-416 that may be utilized for identifying and tracking biological and/or artificial structures 210, 220, 230 by an artificial intelligence segmentation module 140 based on ultrasound operator interaction, in accordance with various embodiments. Referring to FIG. 4, there is shown a flow chart 400 comprising exemplary steps 402 through 416. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 402, an ultrasound system 100 acquires an ultrasound image 200. For example, the ultrasound system 100 may acquire an ultrasound image with an ultrasound probe 104 positioned at a scan position over region of interest.

At step 404, a signal processor 132 of the ultrasound system 100 segments the acquired ultrasound image 200 with artificial intelligence to identify at least one biological and/or artificial structure 210, 220, 230. For example, an artificial intelligence segmentation processor 140 of the signal processor 132 may be configured to analyze the ultrasound image 200 acquired at step 402 to identify and segment biological and/or artificial structures 210, 220, 230. The artificial intelligence segmentation processor 140 may include artificial intelligence image analysis algorithms, one or more deep neural networks (e.g., a convolutional neural network) and/or may utilize any suitable form of artificial intelligence image analysis techniques or machine learning processing functionality configured to analyze acquired ultrasound images to identify and segment biological and/or artificial structures 210, 220, 230 in the ultrasound image 200.

At step 406, a signal processor 132 of the ultrasound system 100 may label 212, 214, 218, 222, 224, 226, 232, 236 the at least one biological and/or artificial structure 210, 220, 230 identified with the artificial intelligence. For example, the artificial intelligence segmentation processor 140 of the signal processor 132 may be configured to label 212, 214, 218, 222, 224, 226, 232, 236 the identified and segmented structures identified at step 404. The labels 212, 214, 218, 222, 224, 226, 232, 236 may include colorizing 218 the pixels of the segmented structure 210, outlining the edges 212, 222, 226, 232, 236 of the segmented structure 210, 220, 230, identifying the segmented structure 210, 220 by a number 214, 224 or letter, and/or any suitable label for drawing attention to one or more structures identified and segmented by the artificial intelligence segmentation processor 140. In various embodiments, the labels of different structures 210, 220, 230 may be different colors and/or different label types. The labels may be overlaid on the ultrasound image 200.

At step 408, the signal processor 132 of the ultrasound system 100 may present the ultrasound image 200 having the labeled 212, 214, 218, 222, 224, 226, 232, 236 at least one biological and/or artificial structure 210, 220, 230. For example, the artificial intelligence segmentation processor 140 of the signal processor 132 may be configured to present the labeled structure(s) 210, 220, 230 at a display system 134 of the ultrasound system 100.

At step 410, the signal processor 132 of the ultrasound system 100 receives a user instruction selecting at least one target, each of the at least one target corresponding with at least one of the labeled structures 210, 220, 230. For example, the artificial intelligence segmentation processor 140 of the signal processor 132 may receive an operator selection, via user input device 130, of one or more labeled structures 210, 220 to be tracked in subsequently acquired ultrasound images 200. The selection of a labeled structure 210, 220, 230 identifies a target to track in subsequent ultrasound images 200. The ultrasound operator may provide a voice command, probe gesture, button depression, or the like that instructs the artificial intelligence segmentation processor 140 to select labeled structures 210, 220, 230 to track and/or deselect labeled structures 210, 220, 230 from being identified in subsequent ultrasound images 200. The selection may include selecting multiple targets to be tracked and/or instructing the artificial intelligence segmentation processor 140 to merge the targets to be tracked in subsequent ultrasound images. The artificial intelligence segmentation processor 140 may modify the image identification, segmentation, labeling, and/or tracking parameters dynamically in response to the user instructions received via the user input device 130.

At step 412, the signal processor 132 of the ultrasound system 100 tracks the selected at least one target 210, 220, 230 by identifying the at least one selected target 210, 220, 230 in subsequent ultrasound images 200 acquired continuously. For example, the artificial intelligence segmentation processor 140 of the signal processor 132 may continue to selectively label and/or otherwise identify the biological and/or artificial structures 210, 220, 230 selected as targets at step 410. The identification may include colorizing 218 the pixels of the target structure 210, outlining the edges 212, 222, 226, 232, 236 of the target structure 210, 220, 230, identifying the target structure 210, 220 by text, and/or any suitable identification for drawing attention to the one or more targets 210, 220, 230 selected by the ultrasound operator.

At step 414, the signal processor 132 of the ultrasound system 100 may provide user feedback 300 based on the location of the tracked at least one target 210, 220, 230 in the continuously acquired ultrasound images 200. For example, the artificial intelligence segmentation processor 140 of the signal processor 132 may be configured to provide audible, visual, and/or physical feedback 300 if a tracked target 210, 220, 230 is approaching an image boundary.

At step 416, the process 400 may end when the ultrasound procedure is finished.

Aspects of the present disclosure provide a method 400 and system 100 for facilitating interaction by an ultrasound operator with an artificial intelligence segmentation module 140 configured to identify and track biological and/or artificial structures 210, 220, 230 in ultrasound images 200. In accordance with various embodiments, the method 400 may comprise acquiring 402, by an ultrasound system 100, an ultrasound image. The method 400 may comprise segmenting 404, by at least one processor 132, 140 executing artificial intelligence, the ultrasound image to identify at least one structure 210, 220, 230 in the ultrasound image. The method 400 may comprise labeling 406, by the at least one processor 132, 140, the at least one structure 210, 220, 230 in the ultrasound image to create a labeled ultrasound image 200. The method 400 may comprise presenting 408, by the at least one processor 132, 140, the labeled ultrasound image 200 at a display system 134. The method 400 may comprise receiving 410, by the at least one processor 132, 140, a user selection of at least one target 210, 220, 230, each of the at least one target 210, 220, 230 corresponding with at least one labeled structure 210, 220, 230. The method 400 may comprise tracking 412, by the at least one processor 132, 140, the selected at least one target 210, 220, 230 by identifying the selected at least one target 210, 220, 230 in subsequently acquired ultrasound images 200.

In certain embodiments, the subsequently acquired ultrasound images 200 are acquired continuously. In various embodiments, the at least one structure 210, 220, 230 comprises one or both of a biological structure or an artificial structure. In a representative embodiment, the user selection is provided via one of: a voice command, an ultrasound probe gesture, or a user input control attached to or integrated with an ultrasound probe 104. In an exemplary embodiment, the labeling 406 comprises one or more of: colorizing pixels 218 of the at least one structure 210, 220, 230, outlining edges 212, 222, 232, 226, 236 of the at least one structure 210, 220, 230, and providing a number 214, 234, a letter, or text associated with the at least one structure 210, 220, 230. In certain embodiments, the identifying the selected at least one target comprises one or more of: colorizing pixels 218 of the at least one target 210, 220, 230, outlining edges 212, 222, 232, 226, 236 of the at least one target 210, 220, 230, and providing a number 214, 234, a letter, or text associated with the at least one target 210, 220, 230. In various embodiments, the labeling 406 is based on a plurality of confidence levels of the segmenting 404 performed by the at least one processor 132, 140 executing the artificial intelligence, and a different label 212, 214, 218, 222, 224, 226, 232, 236 is provided for each of the plurality of confidence levels. In certain embodiments, the method 400 may comprise providing 414, by the at least one processor 132, 140, user feedback 300 based on location of the selected at least one target 210, 220, 230 in the subsequently acquired ultrasound images 200. The user feedback 300 may be one or more of audio feedback, visual feedback, and physical feedback.

Various embodiments provide a system 100 for facilitating interaction by an ultrasound operator with an artificial intelligence segmentation module 140 configured to identify and track biological and/or artificial structures 210, 220, 230 in ultrasound images 200. The system 100 may comprise an ultrasound system 100, at least one processor 132, 140, a user input device 130, and a display system 134. The ultrasound system 100 may be configured to acquire an ultrasound image. The at least one processor 132, 140 may be configured to segment the ultrasound image with artificial intelligence to identify at least one structure 210, 220, 230 in the ultrasound image. The at least one processor 132, 140 may be configured to label 212, 214, 218, 222, 224, 226, 232, 236 the at least one structure 210, 220, 230 in the ultrasound image to create a labeled ultrasound image 200. The at least one processor 132, 140 may be configured to present the labeled ultrasound image 200 at the display system 134. The at least one processor 132, 140 may be configured to receive a user selection of at least one target 210, 220, 230, each of the at least one target 210, 220, 230 corresponding with at least one labeled structure. The at least one processor 132, 140 may be configured to track the selected at least one target 210, 220, 230 by identifying the selected at least one target 210, 220, 230 in subsequently acquired ultrasound images 200. The user input device 130 may be configured to receive the user selection of the at least one target 210, 220, 230 and provide the user selection to the at least one processor 132, 140. The display system 134 may be configured to present the labeled ultrasound image 200 and the subsequently acquired ultrasound images 200 identifying the selected at least one target 210, 220, 230.

In a representative embodiment, the ultrasound system 100 is configured to continuously acquire the subsequently acquired ultrasound images 200. In an exemplary embodiment, the at least one structure 210, 220, 230 comprises one or both of a biological structure or an artificial structure. In certain embodiments, the user selection is provided to the user input device 130 via one of: a voice command, an ultrasound probe gesture, or a user input control attached to or integrated with an ultrasound probe 104. In various embodiments, the at least one processor 132, 140 is configured to label 212, 214, 218, 222, 224, 226, 232, 236 the at least one structure 210, 220, 230 by one or more of: colorizing pixels 218 of the at least one structure 210, 220, 230, outlining edges 212, 222, 232, 226, 236 of the at least one structure 210, 220, 230, and providing a number 214, 234, a letter, or text associated with the at least one structure 210, 220, 230. In a representative embodiment, the at least one processor 132, 140 is configured to identify the selected at least one target 210, 220, 230 by one or more of: colorizing pixels 218 of the at least one target 210, 220, 230, outlining edges 212, 222, 232, 226, 236 of the at least one target 210, 220, 230, and providing a number 214, 234, a letter, or text associated with the at least one target 210, 220, 230. In an exemplary embodiment, the at least one processor 132, 140 is configured to provide user feedback 300 based on location of the selected at least one target 210, 220, 230 in the subsequently acquired ultrasound images 200. The user feedback 300 may be one or more of audio feedback, visual feedback, and physical feedback.

Certain embodiments provide a non-transitory computer readable medium having stored thereon, a computer program having at least one code section. The at least one code section is executable by a machine for causing the machine to perform steps 400. The steps 400 may comprise receiving 402 an ultrasound image. The steps 400 may comprise segmenting 404 the ultrasound image with artificial intelligence to identify at least one structure 210, 220, 230 in the ultrasound image. The steps 400 may comprise labeling 406 the at least one structure 210, 220, 230 in the ultrasound image to create a labeled ultrasound image 200. The steps 400 may comprise presenting 408 the labeled ultrasound image 200 at a display system 134. The steps 400 may comprise receiving 410 a user selection of at least one target 210, 220, 230, each of the at least one target 210, 220, 230 corresponding with at least one labeled structure 210, 220, 230. The steps 400 may comprise tracking 412 the selected at least one target 210, 220, 230 by identifying the selected at least one target 210, 220, 230 in subsequently received ultrasound images 200.

In an exemplary embodiment, the subsequently received ultrasound images 200 are received continuously. In a representative embodiment, the labeling 406 comprises one or more of: colorizing pixels 218 of the at least one structure 210, 220, 230, outlining edges 212, 222, 232, 226, 236 of the at least one structure 210, 220, 230, and providing a number 214, 234, a letter, or text associated with the at least one structure 210, 220, 230. In various embodiments, the identifying the selected at least one target 210, 220, 230 comprises one or more of: colorizing pixels 218 of the at least one target 210, 220, 230, outlining edges 212, 222, 232, 226, 236 of the at least one target 210, 220, 230, and providing a number 214, 234, a letter, or text associated with the at least one target 210, 220, 230. In certain embodiments, the steps 400 may comprise providing user feedback 300 based on location of the selected at least one target 210, 220, 230 in the subsequently received ultrasound images 200. The user feedback 300 may be one or more of audio feedback, visual feedback, and physical feedback.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for facilitating interaction by an ultrasound operator with an artificial intelligence segmentation module configured to identify and track biological and/or artificial structures in ultrasound images.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by an ultrasound system, an ultrasound image;
segmenting, by at least one processor executing artificial intelligence, the ultrasound image to identify at least one structure in the ultrasound image;
labeling, by the at least one processor, the at least one structure in the ultrasound image to create a labeled ultrasound image;
presenting, by the at least one processor, the labeled ultrasound image at a display system;
receiving, by the at least one processor, a user selection of at least one target, each of the at least one target corresponding with at least one labeled structure; and
tracking, by the at least one processor, the selected at least one target by identifying the selected at least one target in subsequently acquired ultrasound images.

2. The method of claim 1, wherein the subsequently acquired ultrasound images are acquired continuously.

3. The method of claim 1, wherein the at least one structure comprises one or both of a biological structure or an artificial structure.

4. The method of claim 1, wherein the user selection is provided via one of:
a voice command,
an ultrasound probe gesture, or
a user input control attached to or integrated with an ultrasound probe.

5. The method of claim 1, wherein the labeling comprises one or more of:
colorizing pixels of the at least one structure,
outlining edges of the at least one structure, and
providing a number, a letter, or text associated with the at least one structure.

6. The method of claim 1, wherein the identifying the selected at least one target comprises one or more of:
colorizing pixels of the at least one target,
outlining edges of the at least one target, and
providing a number, a letter, or text associated with the at least one target.

7. The method of claim 1, wherein the labeling is based on a plurality of confidence levels of the segmenting performed by the at least one processor executing the artificial intelligence, and wherein a different label is provided for each of the plurality of confidence levels.

8. The method of claim 1, comprising providing, by the at least one processor, user feedback based on location of the selected at least one target in the subsequently acquired ultrasound images, wherein the user feedback is one or more of audio feedback, visual feedback, and physical feedback.

9. A system comprising:
an ultrasound system configured to acquire an ultrasound image;
at least one processor configured to:
segment the ultrasound image with artificial intelligence to identify at least one structure in the ultrasound image;
label the at least one structure in the ultrasound image to create a labeled ultrasound image;
present the labeled ultrasound image at a display system;
receive a user selection of at least one target, each of the at least one target corresponding with at least one labeled structure; and
track the selected at least one target by identifying the selected at least one target in subsequently acquired ultrasound images;
a user input device configured to receive the user selection of the at least one target and provide the user selection to the at least one processor; and
the display system configured to present the labeled ultrasound image and the subsequently acquired ultrasound images identifying the selected at least one target.

10. The system of claim 9, wherein the ultrasound system is configured to continuously acquire the subsequently acquired ultrasound images.

11. The system of claim 9, wherein the at least one structure comprises one or both of a biological structure or an artificial structure.

12. The system of claim 9, wherein the user selection is provided to the user input device via one of:
a voice command,
an ultrasound probe gesture, or
a user input control attached to or integrated with an ultrasound probe.

13. The system of claim 9, wherein the at least one processor is configured to label the at least one structure by one or more of:
colorizing pixels of the at least one structure,
outlining edges of the at least one structure, and
providing a number, a letter, or text associated with the at least one structure.

14. The system of claim 9, wherein the at least one processor is configured to identify the selected at least one target by one or more of:
colorizing pixels of the at least one target,
outlining edges of the at least one target, and
providing a number, a letter, or text associated with the at least one target.

15. The system of claim 9, wherein the at least one processor is configured to provide user feedback based on location of the selected at least one target in the subsequently acquired ultrasound images, wherein the user feedback is one or more of audio feedback, visual feedback, and physical feedback.

16. A non-transitory computer readable medium having stored thereon, a computer program having at least one code section, the at least one code section being executable by a machine for causing the machine to perform steps comprising:
receiving an ultrasound image;
segmenting the ultrasound image with artificial intelligence to identify at least one structure in the ultrasound image;
labeling the at least one structure in the ultrasound image to create a labeled ultrasound image;
presenting the labeled ultrasound image at a display system;
receiving a user selection of at least one target, each of the at least one target corresponding with at least one labeled structure; and
tracking the selected at least one target by identifying the selected at least one target in subsequently received ultrasound images.

17. The non-transitory computer readable medium of claim 16, wherein the subsequently received ultrasound images are received continuously.

18. The non-transitory computer readable medium of claim 16, wherein the labeling comprises one or more of:
colorizing pixels of the at least one structure,
outlining edges of the at least one structure, and
providing a number, a letter, or text associated with the at least one structure.

19. The non-transitory computer readable medium of claim 16, wherein the identifying the selected at least one target comprises one or more of:
colorizing pixels of the at least one target,
outlining edges of the at least one target, and
providing a number, a letter, or text associated with the at least one target.

20. The non-transitory computer readable medium of claim 16, comprising providing user feedback based on location of the selected at least one target in the subsequently received ultrasound images, wherein the user feedback is one or more of audio feedback, visual feedback, and physical feedback.
